(19) United States
(12) Patent Application Publication
(10) Pub. No.: US 2010/0003326 A1
(43) Pub. Date: Jan. 7, 2010

(54) DRUG DELIVERY DEVICE COMPRISING A PHARMACEUTICALLY OR BIOLOGICALLY ACTIVE COMPONENT AND AN INFRARED ABSORBING COMPOUND

(75) Inventors: Mikael Allan Myriam Eugene Vertommen, Eindhoven (NL); Dirk Theodorus Andreas Van Asseldonk, Eindhoven (NL); Maria Francisca Kemmere, Helmond (NL); JohannesTheodorus Faustinus Keurentjes, Helmond (NL)

Correspondence Address: SUGHRUE MION, PLLC, 2100 PENNSYLVANIA AVENUE, N.W., SUITE 800, WASHINGTON, DC, 20037, US

(73) Assignee: DOLPHYS TECHNOLOGIES B.V., Eindhoven (NL)

(21) Appl. No.: 12/376,084

(22) PCT Filed: Jul. 30, 2007
(86) PCT No.: PCT/NL07/50381

§ 371 Date: Mar. 25, 2009

Related U.S. Application Data
(60) Provisional application No. 60/834,492, filed on Aug. 1, 2006.

(51) Int. Cl. A61K 9/00

(52) U.S. Cl. 424/486

(57) ABSTRACT

The present invention relates to a drug delivery device for implantation in a mammalian body comprising a thermo-sensitive polymeric material, a pharmaceutically or biologically active component and an infrared absorbing compound. The present invention also relates to a method for releasing an active compound from a drug delivery device, the drug delivery device comprising a thermo-sensitive polymeric material, a pharmaceutically or biologically active component and an infrared absorbing compound, wherein the drug delivery device is exposed to infrared irradiation thereby crossing a phase transition.

### TECHNICAL FIELD OF THE INVENTION

The present invention relates to a drug delivery device which delivers a pharmaceutically or biologically active component in a controlled manner, wherein the delivery of the pharmaceutically or biologically active component is triggered on demand or wherein the delivery of the pharmaceutically or biologically active component is reversible, that is that, once induced, the delivery can be stopped and subsequently initiated again thereby providing a repeatable delivery. The delivery is controlled by an external impulse.

More in particular, the present invention relates to a drug delivery device comprising a thermo-sensitive polymeric material comprising a pharmaceutically or biologically active component and an additive sensitive to infrared irradiation, wherein delivery of the pharmaceutically or biologically active component is activated by supplying energy from an external source of infrared irradiation. The drug delivery device is suitable for implantation in a mammalian body, for incorporation into a medical device, like a prosthesis, an artificial organ, a catheter, a surgical mesh and the like.

### BACKGROUND OF THE INVENTION

Materials such as polymers may have a barrier effect against diffusion of a pharmaceutically or biologically active component. For example, WO 2004/113422, incorporated by reference herein, discloses a drug delivery device, in particular for implantation in a mammalian body, comprising a solid polymeric material having a barrier effect against diffusion of chemical substances or against electron transport, in particular a pharmaceutically or biologically active component, and having a glass transition temperature within the range of about 0° to about 90° C., and a pharmaceutically or biologically active component, wherein the characteristics of diffusion or transport of the active component can be modified in a reversible manner by supplying energy from an energy source. The energy source is preferably selected from the group consisting of light waves (e.g. infrared irradiation), sound waves, microwaves, electric current, electrical, magnetic or radioactive irradiation or a combination thereof. In operation, energy is absorbed which results in a glass transition of the solid polymeric material, i.e. from the glassy to the rubbery state, thereby enhancing the delivery of the pharmaceutically or biologically active component. When the supply of energy is discontinued, the solid polymeric material is subjected again to a glass transition so that the delivery of the pharmaceutically or biologically active component is substantially decreased or even shut down completely.

U.S. Pat. No. 6,428,811 and U.S. Pat. No. 6,645,517, incorporated by reference herein, disclose a thermally sensitive polymer-particle composite that absorbs electromagnetic irradiation. The absorbed energy is used to trigger the delivery of a pharmaceutically or biologically active component. The composite comprises a thermo-sensitive polymer, a pharmaceutically or biologically active component and light-absorbing particles, in particular metal nanoshells. The thermo-sensitive polymer has preferably a lower critical solution temperature (LCST) that is slightly higher than the normal body temperature of the patient. The electromagnetic irradiation is preferably near infrared irradiation. The thermally sensitive polymer-particle composite enables delivery of the active component on demand. However, the disadvantage of the composite disclosed in U.S. Pat. No. 6,428,811 and U.S. Pat. No. 6,645,517 is in particular that elusive metal nanoshells (cf. U.S. Pat. No. 6,344,272, incorporated by reference herein) must be used. These metal nanoshells are particles having a non-conducting core, preferably monodisperse colloidal silica, and a conducting shell layer, preferably consisting of gold. By varying the ratio of the diameter of the core and the thickness of the shell layer, the absorption maxima of the metal nanoshells can be tuned within the region of about 500 nm to about 1200 nm. The metal nanoshells are capable of absorbing near infrared irradiation which results in so-called “plasmon resonance” which causes a temperature rise. However, these metal nanoshells are difficult and laborious to prepare and to the applicant's knowledge are not commercially available

U.S. Pat. No. 6,916,866, incorporated by reference herein, discloses a laser-absorbing molding composition comprising a laser transparent thermoplastic material and a compound that is capable of absorbing infrared irradiation. The laser-absorbing compositions are used for the production of molded parts that are joined to other molded parts by laser beam welding.

The present invention provides a drug delivery system that does not have the disadvantages of the prior art drug delivery systems discussed above.

### SUMMARY OF THE INVENTION

Accordingly, the present invention provides a drug delivery device, in particular for implantation in a mammalian body, comprising a thermo-sensitive polymeric material, a pharmaceutically or biologically active component and an infrared absorbing compound.

The present invention also relates to a method for releasing an active compound, from a drug delivery device, in particular for implantation in a mammalian body, the drug delivery device comprising a thermo-sensitive polymeric material, a pharmaceutically or biologically active component and an infrared absorbing compound, wherein the drug delivery device is exposed to infrared irradiation.

### DETAILED DESCRIPTION OF THE INVENTION

In this document and in its claims, the verb “to comprise” and its conjugations are used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

Within the framework of the present invention, the term “infrared irradiation” is to be understood to encompass a wavelength of 500 nm-1000 μm, preferably 700 to 1200 nm, and more preferably 700-1050 nm, the latter two ranges also being known as the near-infrared regions of the electromagnetic spectrum.

Also within the framework of the present invention, the term “pharmaceutically or biologically active component” is to be understood as a component that provides, either directly or indirectly, a certain pharmaceutical or biological action, preferably to a mammalian body. Hence, according to the invention, an active component may be a medicament or pharmaceutical that has a direct biological effect on a mammalian body.

Further, within the framework of the invention, the glass transition temperature T_{g} is not limited to the glass transition temperature of the thermo-sensitive polymeric material per se. The glass transition temperature T_{g} may also denote the glass transition temperature of e.g. the combination of the thermo-sensitive polymeric material, the pharmaceutically or biologically active component and the infrared absorbing compound, i.e. that the glass transition temperature as used herein does not refer to the glass transition temperature of the polymeric material per se, unless otherwise stated. In addition, the drug delivery device according to the present invention may comprise further components or additives that influence the glass transition temperature. Furthermore, when in use, for example as an implant, the drug delivery device according to the present invention may be in contact with further components, e.g. body fluids which are usually aqueous in nature, that effect the glass transition temperature of the drug delivery device. Consequently, the glass transition temperature T_{g} should be interpreted as the glass transition temperature of the thermo-sensitive part or region of the drug delivery device according to the present invention which comprises the thermo-sensitive polymeric material. As will become apparent below, the thermo-sensitive part or region of the drug delivery device according to the present invention may constitute a core, a sublayer or an outer layer of the drug delivery device. Preferably, the thermo-sensitive part or region is an outer layer, more preferably the outer layer, of the drug delivery device according to the invention. However, it will be apparent to the person skilled in the art that it is not necessary that the thermo-sensitive part or region is the outer layer, but that it may be surrounded by another layer as the outer layer, provided that this other-outer-layer is permeable for the pharmaceutically or biologically active component.

Likewise, the lower critical solution temperature (LCST) as used in this document relates to the LCST of the thermo-sensitive part or region of the drug delivery device.

Additionally, the melt transition as used in this document relates to the melt transition of the thermo-sensitive part or region of the drug delivery device.

Additionally, an infrared absorbing compound is to be understood as a compound or structure capable of providing a temperature rise upon irradiation with infrared irradiation, in particular infrared light having a wavelength of 700 to 1200 nm and more in particular infrared light having a wavelength of 700-1050 nm. Preferably the infrared absorbing compound is a compound comprising carbon. More preferably the infrared absorbing compound is selected from the group consisting of organic compounds, organometallic compounds and carbon compounds including carbon black (also known in the art as “acetylene black”), carbon nanotubes, carbon nanocages, carbon nanorods and the like.

The advantages of the present invention are obtained by employing a drug delivery device of which the release characteristics are modified on demand and/or are modified in a reversible manner by supplying infrared irradiation that optionally differs as a function of time. The release characteristics are modified by employing thermo-sensitive polymeric materials in combination with infrared absorbing compounds, wherein the infrared absorbing compounds provide heat upon irradiation with infrared light and cause a change in the release characteristics of the drug delivery device. The thermo-sensitive part or region of the drug delivery device has in particular a T_{g} within a certain range, wherein upon irradiating the drug delivery device with infrared irradiation, the infrared absorbing compound comprised by the drug delivery device absorbs said infrared irradiation thereby radiating heat which results in heating of the thermo-sensitive part or region above its T_{g} and in an enhanced release of the active component that is present. According to the invention, it is preferred that the thermo-sensitive polymeric part or region of the drug delivery device has a T_{g} in the range of about 0° to about 90° C., more preferably about 30° to about 90° C.

Alternatively, the thermo-sensitive part or region can absorb heat upon irradiating the drug delivery device which results in heating the thermo-sensitive part or region to a temperature above the LCST of the thermo-sensitive part or region. Similarly, upon absorption of heat the thermo-sensitive part or region can pass a melt transition which results in an enhanced release of the active component. As will be understood by the person skilled in that art, the thermo-sensitive part or region may pass more than one transition temperature.

When the drug delivery device according to a preferred embodiment of the present invention is implanted in the body of a mammal, it is surrounded by an aqueous environment, e.g. body fluids. This environment may influence the T_{g} of the thermo-sensitive part or region of the drug delivery device since aqueous components such as water may be absorbed by the drug delivery device, in particular by the thermo-sensitive polymeric material. In addition, infrared irradiation is less absorbed by the aqueous environment than by the thermo-sensitive part or region of the drug delivery device. This is also known as the “water gap” or “therapeutic window” of the absorption spectrum of tissue. Consequently, irradiation with infrared does not have any harmful effect to a patient.

An advantage of the drug delivery device and method according to the present invention is that the release characteristics can be reversibly modified in such a way that the release of the active component occurs repeatedly and reproducibly. In this way, the release of an active component can be controlled as a function of time. Another advantage of the present invention is that an active component can be delivered on demand.

Depending on the therapeutic or diagnostic applications and the pharmaceutically or biologically active component concerned, the drug delivery device according to the invention can be made in various ways. According to a first, preferred embodiment, the pharmaceutically or biologically active component and the infrared absorbing compound are mixed, preferably in a homogeneous manner, with the thermo-sensitive polymeric material and form all together the thermo-sensitive part or region of the drug delivery device.

According to a second, preferred embodiment, the pharmaceutically or biologically active component is comprised by the core of the drug delivery device and this core is surrounded by a layer comprising the thermo-sensitive polymeric material and the infrared absorbing compound. In this second, preferred embodiment, the layer comprising the thermo-sensitive polymeric material and the infrared absorbing compound forms the thermo-sensitive part or region of the drug delivery device.

In a third, preferred embodiment, the core of the drug delivery device comprises the infrared absorbing compound and the pharmaceutically or biologically active component, said core being surrounded by a layer comprising the thermo-sensitive polymeric material. In this third, preferred embodiment the thermo-sensitive part or region of the drug delivery device is formed by this layer comprising the thermo-sensitive polymeric material.

In a fourth, preferred embodiment, the core of the drug delivery device comprises the infrared absorbing compound, said core being surrounded by a layer comprising the pharmaceutically or biologically active component and the thermo-sensitive polymeric material. Consequently, in this embodiment the thermo-sensitive part or region of the drug delivery device is formed by this layer comprising the combination of the pharmaceutically or biologically active component and the thermo-sensitive polymeric material.

Consequently, the drug delivery device according to the present invention may comprise:
(a) a core comprising a thermo-sensitive polymeric material, a pharmaceutically or biologically active component and an infrared absorbing compound and optionally an outer layer, wherein said outer layer is permeable for said pharmaceutically or biologically active component;
(b) a core comprising a pharmaceutically or biologically active component and an outer layer comprising a thermo-sensitive polymeric material and an infrared absorbing compound, wherein said outer layer is optionally surrounded by an other layer, said other layer being permeable for said pharmaceutically or biologically active component;
(c) a core comprising a pharmaceutically or biologically active component and a infrared absorbing compound and an outer layer comprising the thermo-sensitive polymeric material, wherein said outer layer is optionally surrounded by an other layer, said other layer being permeable for said pharmaceutically or biologically active component; and
(d) a core comprising the infrared absorbing compound and an outer layer comprising the thermo-sensitive polymeric material and the pharmaceutically or biologically active component, wherein said outer layer is optionally surrounded by an other layer, said other layer being permeable for said pharmaceutically or biologically active component

In order to protect the tissues and/or the organs in which the drug delivery device is present from the heat produced by the drug delivery device comprising the infrared absorbing compound when irradiated by infrared irradiation, it is preferable according to a fifth preferred embodiment of the invention that the drug delivery device comprises a layer surrounding the core and optionally the outer layer as disclosed above. This layer is preferably an enveloping material that is thermally insulating and is obviously permeable for the pharmaceutically or biologically active component. In addition, this enveloping material counteracts cooling of the outer part of the thermo-sensitive part or region of the drug delivery device. Too-much cooling is undesired since it causes the outer part of the thermo-sensitive part or region to remain having a temperature below its transition temperature, e.g. T_{g}, CST or melt, during irradiation with IR, resulting in a far less enhanced delivery of the pharmaceutically or biologically active component during irradiation. For example, if the drug delivery device is implanted in a mammal, e.g. a human, the mammalian tissue may cause that the outer part of the thermo-sensitive part or region is maintained at the normal body temperature of the mammal so that, even upon irradiation with infrared irradiation, a crossing of the transition temperature is prevented. Obviously, when irradiation with infrared irradiation is deliberately interrupted, the enveloping material should be capable to cool sufficiently to decrease or to stop the release of the active component.

Accordingly, this fifth preferred embodiment of the present invention comprises a drug delivery device, in particular for implantation in a mammalian body, comprising:
(i) a central region comprising any one of embodiments (a)-(d) disclosed above; and
(ii) an enveloping material surrounding the central region, said enveloping material being thermally insulating and permeable for the active component.

The central region of this preferred embodiment may have different structures as disclosed above.

Alternatively, according to a sixth preferred embodiment, the drug delivery device according to the present invention comprises a central region comprising an inert support, said central region having a first layer comprising any one of embodiments (a)-(d) disclosed above (so that the “core” as defined in any one of embodiments (a)-(d) constitutes a first sublayer and the “outer layer” as defined in any one of embodiments (a)-(d) constitutes a second sublayer surrounding said first sublayer), wherein the first layer is coated or covered with the enveloping material.

According to the invention, the embodiments (a)-(d) may occur as a single entity within the drug delivery device. However, as will be apparent to the skilled person, the drug delivery device may comprise multiple entities of embodiments (a)-(d). For example, the drug delivery device may comprise a core region comprising a multitude of cores comprising a pharmaceutically or biologically active component, wherein the core region is surrounded by an outer layer comprising a thermo-sensitive polymeric material and an infrared absorbing compound, wherein said outer layer is optionally surrounded by an other layer, said other layer being permeable for said pharmaceutically or biologically active component. Alternatively, the drug delivery device may comprise a multitude of said cores wherein each core is surrounded by said outer layer.

The enveloping material preferably creates an aqueous environment that surrounds the drug delivery device. The enveloping material may be selected from a wide range of materials and structures and include e.g. the wall of a catheter (wherein the drug delivery device is incorporated in the wall of the catheter), a modification of the surface of the outer layer (for example, a surface having indentations), a porous web-like material, either woven or non-woven, or a hydrogel.

Examples of a web-like enveloping material include bandage materials, wound dressings and sticking plasters.

Hydrogels are three dimensional networks of hydrophilic polymers in which a large amount of water is present. In general the amount of water present in a hydrogel is at least 20 weight percent of the total weight of the dry polymer. The most characteristic property of these hydrogels is that they swell in the presence of water and shrink in the absence of water. The extent of swelling (equilibrium water content) is determined by the nature (mainly the hydrophilicity) of the polymer chains and the crosslinking density. Polymers that can be used for manufacturing hydrogels are well known in the art and are for example disclosed in U.S. Pat. No. 2,340,110, U.S. Pat. No. 2,340,111, U.S. Pat. No. 2,533,635, U.S. Pat. No. 2,798,053, U.S. Pat. No. 3,940,351, U.S. Pat. No. 4,062,817, U.S. Pat. No. 5,034,486, U.S. Pat. No. 5,034,487, U.S. Pat. No. 5,034,488 and U.S. Pat. No. 5,468,797, all incorporated by reference herein. Another type of hydrogel is based on supramolecular host-guest chemistry, e.g. a hydrogel made by reversible cross-linking of an acryl amide polymer having pendant adamantyl groups with a cyclodextrine dimer as disclosed by Kretschmann et al., Angew. Chem. Int. Ed. Engl. 45, 4361-4365, 2006.

According to a seventh preferred embodiment of the present invention, the drug delivery device comprises a material comprising the infrared absorbing compound, said material being permeable for the pharmaceutically or biologically active component, and particles comprising the thermo-sensitive polymeric material as well as the pharmaceutically or biologically active component, wherein said material comprising the infrared absorbing compound and being permeable for the active component is heated by supplying infrared irradiation. Preferably, said material is coated with an enveloping material being thermally insulating and permeable for the pharmaceutically or biologically active component. The advantage of this seventh preferred embodiment is that the environment in which the drug delivery device is present and in which the method is thus employed, in particular the physiological environment of the mammalian body, can be protected from changes in the drug delivery device that are a consequence of the supply of infrared irradiation. One example of this is protection from a rise in the temperature of the drug delivery device.

In addition, the use of an enveloping material being thermally insulating and permeable for the active compound has as further advantages that it can counteract fouling of the thermo-sensitive polymeric material. It can further provide more strength to the drug delivery device and provide flexibility thereto and inhibits negative influences from sarcophagus and aggressive bacteria. It further provides the option that thermo-sensitive polymeric materials can be used which are less biocompatible (rejections) provided that the enveloping material is sufficiently biocompatible.

Research has revealed that particular good results are obtained when a thermo-sensitive polymeric material is employed comprising poly(lactic acid-co-glycolic acid), polymethyl (meth)acrylate, poly(N-isopropylacrylamide), poly(N,N-dimethylacrylamide), a nylon, or a polymer or a copolymer comprising a monomer selected from the group consisting of a hydroxyl alkanoate wherein the alkyl group comprises 1 to 12 carbon atoms, lactide, glycolide, ε-caprolactone, 1,4-dioxane-2-one, 1,5-dioxepan-2-one, trimethylene carbonate (1,3-dioxane-2-one) and mixtures thereof, as well as polymers having side-chain crystallinity. Preferably, the copolymer is an alternating or random copolymer or a block copolymer and the block copolymer is preferably a diblock copolymer or a triblock copolymer. Such polymers and copolymers are well known in the art and are for example disclosed in U.S. Pat. No. 2,668,162, U.S. Pat. No. 2,703,316, U.S. Pat. No. 3,636,956, U.S. Pat. No. 3,839,297, U.S. Pat. No. 4,137,921, U.S. Pat. No. 4,157,437, U.S. Pat. No. 4,243,775, U.S. Pat. No. 4,443,430, U.S. Pat. No. 4,830,855, U.S. Pat. No. 5,076,983, U.S. Pat. No. 5,310,865 and U.S. Pat. No. 6,025,458, all incorporated by reference herein. This group of even more preferred thermo-sensitive polymeric materials also includes (meth)acrylic (co)polymers, polyester urethanes, polyester amides, polyether esters such as polyethylene glycol terephthalate-polybutylene terephalate (PEGT/PBT), polyethylene glycol and the natural polymers such as poly(hydralonic acid), iso-sorbide, dextran, collagens and mixtures thereof. Another even more preferred thermo-sensitive polymeric material is also poly(-n-butyl methacrylate) and poly(n-butylmethacrylate-co-methylmethacrylate).

Research has further revealed that particular good results are obtained when an infrared absorbing compound is employed comprising so called “rylenes” (perylenes, terrylenes, quaterrylenes and the like) and carbon compounds, provided that the infrared absorbing compound is capable of absorbing infrared irradiation having a wave length in the range of 500 nm-1000 μm, in particular 700 to 1200 nm n.

Particularly advantageous applications of the present invention are in the field of the release of pharmaceutically or biologically active components including medicaments, diagnostic agents and contrast media for imaging. If the active compound is a medicament, it is preferred that the medicament is selected from the group consisting of chemotherapeutic agents, analgesics and anesthetics, hormonal substances, anti-microbial agents, and anti-arrhythmatic agents.

The present invention also relates to a method for releasing a pharmaceutically or biologically active component from a drug delivery device according to the present invention, wherein it is preferred that the drug delivery device is for implantation in a mammalian body. However, other uses are also suitable and are disclosed in this document as well. Consequently, the present invention also relates to a method for releasing an active compound from a drug delivery device, the drug delivery device comprising a thermo-sensitive polymeric material, a pharmaceutically or biologically active component and an infrared absorbing compound, wherein the drug delivery device is exposed to infrared irradiation.

An important advantage of the method according to the invention is that a pharmaceutically or biologically active component can be repeatedly released from the drug delivery device in a controlled manner as a function of time, depending on the therapeutic or diagnostic needs (reversible release). It is preferred that a medicament selected from the group of chemotherapeutic agents, analgesics and anesthetics, hormonal substances, anti-microbial agents and anti-arrhythmatic agents is used as active component. It is likewise preferred that a diagnostic agent or a contrast medium for imaging is used as active component.

The infrared irradiation can be supplied continuously during the time the delivery of the active component is desired. Alternatively, the infrared irradiation can be supplied pulse-wise or intermittently during the time the delivery of the active component is desired. In this way tissue is not continuously exposed to infrared irradiation. Both of these methods of infrared irradiation can be repeated each time the delivery of the active component is desired. For example, if the active component is an analgesic, a patient in need thereof can induce the release of the analgesic on demand by switching on the infrared source.

According to the present invention, the infrared irradiation is supplied externally with respect to the drug delivery device. According to this invention, the release characteristics of the thermo-sensitive polymeric material are preferably be modified remotely.

According to the invention, besides infrared irradiation, other sources may be employed in combination.

The ultimate temperature increase that occurs within the thermo-sensitive polymeric material is dependent on the intensity and wavelength of the infrared irradiation as will be understood by the person skilled in the art. It will also be dependent on the type of infrared absorbing compound that is incorporated in the drug delivery device. For example, a specific inter-atomic bond of the thermo-sensitive polymer or another additive can be activated by using a specific wavelength.

As is disclosed above, the thermo-sensitive polymeric material experiences a phase transition when the infrared absorbing compounds are irradiated with infrared irradiation, thereby heating the polymeric material. This phase transition may be a glass transition, a crossing of the LCST or a melt transition.

If the phase transition involves a glass transition, experiments have shown that it is preferred that the infrared irradiation is used in such a way that the thermo-sensitive polymeric material is subjected to one or more, more preferably two or more, reversible glass transitions between a glassy state and a rubbery state. It has been found that such transitions, which as such require relatively little energy, can lead to very appreciable modifications in the release characteristics, which, in turn, can lead to a very appreciable increase in the diffusion of an active component present in the thermo-sensitive polymeric material. According to a particularly preferred application, the glass transition or glass transitions occur at a temperature of about 30° to about 90° C., more preferably about 35° to about 80° C., even more preferably about 35 to about 65° C., yet even more preferably about 35 to about 60°, yet even more preferably about 37 to about 55° C. and most preferably about 40° to about 55° C. Experiments show that these temperature ranges are adequate to obtain the particular advantages according to the present invention.

If the phase transition involves a crossing of the LCST, the thermo-sensitive polymeric material is in an expanded state below its LCST. Above its LCST, it is in a collapsed state. Different mechanisms for the release of active components are proposed in the art. For example, the drug delivery device may operate like a valve, wherein at temperatures higher than the transition temperature the active component is released due to aggregation of polymer (side)chains or deswelling of polymer networks. At temperatures below the transition temperature, the release is decreased or stooped due to extension of polymer (side)chains or swelling of the polymeric network. Alternatively, the drug delivery device may comprise nanochannels or nanopores that open or close, depending on the temperature.

According to the invention, the LCST of the thermo-sensitive polymeric material is preferably a temperature of about 30° to about 90° C., more preferably about 35° to about 80° C., even more preferably about 35 to about 65° C., yet even more preferably about 35 to about 60°, yet even more preferably about 37 to about 55° C. and most preferably about 40° to about 55° C. Polymers such as poly(N-isopropylacrylamide) and derivatives thereof are known to have a LCST-transition.

Likewise, the thermo-sensitive polymeric material may cross a melt transition, wherein the melt transition preferably occurs within a temperature range of about 30° to about 90° C., more preferably about 35° to about 80° C., even more preferably about 35 to about 65 C, yet even more preferably about 35 to about 60°, yet even more preferably about 37 to about 55° C. and most preferably about 40° to about 55° C. Melt transitions may for example occur in (co)polymers having side-chain crystallinity which are well known in the art. Reference is made to U.S. Pat. No. 4,830,855, incorporated by reference. Side-chain crystallinity occurs for example in (co)polymers having long pending side chains such as poly(hexadecylmethacrylate).

According to yet another embodiment of the invention, particles comprising the thermo-sensitive polymeric material and the active compound can occur in a form that can be administered by injection, e.g. intravenously or intramuscularly, as is for example disclosed in US 2003/0212148, incorporated by reference herein.

The infrared absorbing compound are preferably compounds that absorb infrared irradiation in the near-infrared region (700-1200 nm, more preferably 700-1050 nm), at least in partial ranges of the near-infrared region, whereas they do not absorb or absorb only weakly in the visible spectrum. Obviously, it also preferred that the infrared absorbing compounds are compatible with the thermo-sensitive polymeric material and other optionally used materials such as additives. Suitable infrared absorbing compounds are known from e.g. M. Matsuoka, Infrared Absorbing Dyes, Plenum Press, New York, 1990. Preferred infrared absorbing compounds are selected from the class consisting of phthalocyanines, naphthalocyanines, rylenes (perylenes, terrylenes, quaterrylenes), (organo)metal complexes, azo dyes, anthraquinones, squaric acid derivatives, immonium dyes, polymethines and derivatives thereof, polyanilines and the carbon compounds mentioned above. More preferably, the infrared absorbing compounds are selected from the class consisting of phthalocyanines, naphthalocyanines and rylenes because of their thermal stability. Infrared absorbing compounds having bulky side groups are even more preferred on account of the improved solubility in thermoplastics materials.

The group of rylenes is well known in the art (cf. for example Y. Geerts et al., J. Mater. Chem. 8, 2357-2369, 1998). According to the invention, the rylene is preferably a quaterrylene, more preferably a quaterrylenebis(dicarboximide), wherein reference is expressly made to U.S. Pat. No. 5,986,099 and the Geerts et al. article mentioned above, both incorporated by reference herein. Very suitable quaterrylenebis(dicarboximide)s are Lumogen® IR 788 and IR 765, available from BASF.

The group of phthalocyanines and naphthalocyanines is also well known in the art. Reference is expressly made to U.S. Pat. No. 6,916,866 which is incorporated by reference herein.

### EXAMPLES

### Example 1a

An object consisting of a pressed circular disc with a diameter of 2.5 cm and thickness of 1.5 mm was mounted in a holder in air at 20° C. The disc consisted of poly(methyl methacrylate), or PMMA, in which 100 ppm of a quaterrylenebis(dicarboximide) Lumogen® IR 788 was dissolved, exhibiting an absorbance of 1.7 a.u. at a wavelength of 785 nm. The disc was irradiated perpendicularly to the surface with a fiber coupled laser diode module (λ: 785 nm, CW) exhibiting a circular Gaussian intensity distribution. The laser beam diverged, the beam radius depending on the distance from the fiber exit (L) according to 2·N.A.·L, in which the numerical aperture (N.A.) is 0.22. The laser power was varied from 110 to 740 mW, the distance between the fiber exit and the object being 45 mm.

The temperature was measured using infrared temperature sensors. A Gaussian surface temperature profile at both the irradiated and non-irradiated side was measured. In the center, a surface temperature rise of 3.0° C. after 10 seconds of irradiation and 11.1° C. after 2 minutes was measured using a laser power of 110 mW. Using a laser power of 740 mW, temperature rises of 15.7° C. after 10 s and 75.3° C. after 2 minutes were obtained. Both the initial heating rate, defined as the slope of the time-temperature graph at t=0 s, and the steady state temperature rise increased linearly with power, and thus intensity.

### Example 1b

An object consisting of a pressed circular disc with a diameter of 2.5 cm and thickness of 0.8 mm was mounted in a holder in air at 20° C. The disc consisted of poly(methyl methacrylate), or PMMA, in which 290 ppm of a quaterrylenebis(dicarboximide) Lumogen® IR 765 was dissolved, exhibiting an absorbance of 2.9 a.u. at a wavelength of 785 nm.

The disc was irradiated perpendicularly to the surface with the same laser diode module as in Example 1a, the laser power being 400 mW and the distance between fiber exit and disc 45 mm. A maximum surface temperature rise of 58° C. was reached within 2 minutes of irradiation, after which the temperature remained constant.

### Example 1c

An object consisting of a solvent cast circular film with a diameter of 2.4 cm and thickness of 80 microns was mounted in a holder in air at 20° C. The film consisted of poly(butyl methacrylate-co-methyl methacrylate), or P(BMA-MMA), with a glass transition (T_{g}) onset of 52° C., in which 3800 ppm of a quaterrylenebis(dicarboximide) was dissolved, exhibiting an absorbance of 1.3 a.u. at a wavelength of 785 nm.

The film was irradiated perpendicularly to the surface with the same laser diode module as in Example 1a, the laser power being 400 mW and the distance between fiber exit and film 55 mm. A maximum surface temperature rise of 62° C. was reached within 10 seconds of irradiation, after which the temperature remained constant.

### Example 1d

An object consisting of a solvent cast circular film with a diameter of 2.4 cm and thickness of 80 μm was mounted in a holder in air at 20° C. The film consisted of poly(butyl methacrylate-co-methyl methacrylate), or P(BMA-MMA), with a glass transition (T_{g}) onset of 52° C. in which 1 wt % of partially exfoliated unmodified single wall carbon nanotubes (CarboLex AP grade, 50-70%, available from Carbolex Inc., Broomal, USA) was incorporated, exhibiting an absorbance of 1.5 a.u. at a wavelength of 785 nm.

The film was irradiated perpendicularly to the surface with the same laser diode module as in Example 1a, the laser power being 400 mW and the distance between fiber exit and film 55 mm. A maximum surface temperature rise of 51° C. was reached within 10 seconds of irradiation, after which the temperature remained constant.

### Example 1e

An object consisting of a pressed circular disc with a diameter of 2.5 cm and thickness of 0.9 mm was mounted in a holder in air at 20° C. The disc consisted of poly(methyl methacrylate), or PMMA, in which 240 ppm of a quaterrylenebis(dicarboximide) Lumogen® IR 788 was dissolved, exhibiting an absorbance of 2.4 a.u. at a wavelength of 785 nm.

The film was irradiated perpendicularly to the surface with an Light Emitting Diode (LED) with an output power of 1.1 W (λₘₐₓ: 780 nm) exhibiting a viewing angle of approximately 60°. The distance between the LED and the sample was 2 mm. A maximum surface temperature rise of 73° C. was reached within 2 minutes of irradiation, after which the temperature remained constant.

### Example 2a

An object consisting of a pressed circular disc with a diameter of 2.5 cm and thickness of 2 mm was placed in a holder in a vessel completely containing 90 mL physiological buffer at 30° C. The disc consisted of P(BMA-MMA) with a T_{g} onset of 52° C. in which 290 ppm of the quaterrylenebis(dicarboximide) Lumogen® IR 788 and 5 wt % ibuprofen were incorporated. Addition of ibuprofen led to a lowering of the T_{g} to about 35° C.

The disc was irradiated perpendicularly to the surface with the same laser diode module as in Example 1a, the laser power being 750 mW and the distance between fiber exit and disc 50 mm. The water layer between sample and disc was approximately 20 mm. A glass lid was placed on the vessel to prevent water evaporation. The vessel was completely filled with water, thus avoiding reflection and refraction effects at an additional air/water interface. The near-infrared irradiation only encountered an air to glass and a glass to water transition. Irradiation was applied in an alternating manner, periods of irradiation of 4 hours being alternated by periods of 20 hours without irradiation.

As a consequence of irradiation, the speed of release of ibuprofen from the disc in each case increased from less than 1×10⁻⁶ g/h to values around 3.9×10⁻⁶ g/h. After stopping irradiation, the release of ibuprofen in each case fell virtually immediately to the base value.

### Example 2b

An object consisting of a compounded strand with a diameter of 2.0 mm and a length of 2.0 cm was mounted in a holder in human plasma at 37° C. The strand consisted of poly(L,DL-Lactide), with a T_{g} onset of 58° C., which was prepared by mixing the polymer with 6000 ppm of a quaterrylenebis(dicarboximide) Lumogen® IR 788, exhibiting an absorbance of 2.4 a.u. at a wavelength of 785 nm and 40 wt % fentanyl. Addition of fentanyl led to a lowering of the T_{g} to about 22° C.

The strand was irradiated perpendicularly to the length axis with the same laser diode module as in Example 1a, for 15 minutes, the laser power set at 750 mW and the distance between the fiber exit and strand being 20 mm.

As a consequence of irradiation, the speed of release of fentanyl from the strand to the human plasma increased from below the detection limit of the GC-MS to 6.9 μg in 15 min.

### Example 3

An object consisting of a compounded strand with a diameter of 2.0 mm and a length of 2.0 cm. The strand consisted of poly(L,DL-Lactide), with a T_{g} onset of 58° C., which was prepared by mixing the polymer with 6000 ppm of a quaterrylenebis(dicarboximide) Lumogen® IR 788, exhibiting an absorbance of 2.4 a.u. at a wavelength of 785 nm and 40 wt % ibuprofen. Addition of ibuprofen led to a lowering of the T_{g} to about −1° C.

The strand was placed subcutaneously in a rat. After 1 day, the strand was irradiated perpendicularly to the length axis with the same laser diode module as in Example 1a, for 15 minutes, the laser power set at 750 mW and the distance between fiber exit and the skin of the rat being 20 mm. The next day this procedure was repeated again.

Blood samples were taken before, during and after triggering on both days. As a consequence of irradiation, the concentration of ibuprofen in the blood plasma increased the first time from below 5 ng/ml (no detection peak of LC-MS/MS was found) to 25 ng/ml and the second time from below 5 ng/ml (no peak was found) to 19 ng/ml.

1. A drug delivery device comprising a thermo-sensitive polymeric material, a pharmaceutically or biologically active component and an infrared absorbing compound said infrared absorbing compound comprising carbon.

2. Drug delivery device according to claim 1, wherein the drug delivery device comprises a thermo-sensitive part or region comprising the thermo-sensitive polymeric material, said thermo-sensitive part or region having a glass transition temperature within the range of about 0° to about 90° C. or a lower critical solution temperature (LCST) within the range of about 0° to about 90° C.

3. Drug delivery device according to claim 1, wherein the drug delivery device comprises a core comprising a thermo-sensitive polymeric material, a pharmaceutically or biologically active component and an infrared absorbing compound and optionally an outer layer, wherein said outer layer is permeable for said pharmaceutically or biologically active component

4. Drug delivery device according to claim 1, wherein the drug delivery device comprises a core comprising a pharmaceutically or biologically active component and an outer layer comprising a thermo-sensitive polymeric material and an infrared absorbing compound, wherein said outer layer is optionally surrounded by an other layer, said other layer being permeable for said pharmaceutically or biologically active component.

5. Drug delivery device according to claim 1, wherein the drug delivery device comprises a core comprising a pharmaceutically or biologically active component and a infrared absorbing compound and an outer layer comprising the thermo-sensitive polymeric material, wherein said outer layer is optionally surrounded by an other layer, said other layer being permeable for said pharmaceutically or biologically active component.

6. Drug delivery device according to claim 1, wherein the drug delivery device comprises a core comprising the infrared absorbing compound and an outer layer comprising the thermo-sensitive polymeric material and the pharmaceutically or biologically active component, wherein said outer layer is optionally surrounded by an other layer, said other layer being permeable for said pharmaceutically or biologically active component.

7. Drug delivery device according to claim 1, comprising:
a central region; and
an enveloping material surrounding the central region, said enveloping material being thermally insulating and permeable for the active component,
wherein the central region is selected from the group consisting of:
(i) a core comprising a thermo-sensitive polymeric material, a pharmaceutically or biologically active component and an infrared absorbing compound and optionally an outer layer, wherein said outer layer is permeable for said pharmaceutically or biologically active component;
(ii) a core comprising a pharmaceutically or biologically active component and an outer layer comprising a thermo-sensitive polymeric material and an infrared absorbing compound, wherein said outer layer is optionally surrounded by an other layer, said other layer being permeable for said pharmaceutically or biologically active component;
(iii) a core comprising a pharmaceutically or biologically active component and a infrared absorbing compound and an outer layer comprising the thermo-sensitive polymeric material, wherein said outer layer is optionally surrounded by an other layer, said other layer being permeable for said pharmaceutically or biologically active component; and
(iv) a core comprising the infrared absorbing compound and an outer layer comprising the thermo-sensitive polymeric material and the pharmaceutically or biologically active component, wherein said outer layer is optionally surrounded by an other layer, said other layer being permeable for said pharmaceutically or biologically active component.

8. Drug delivery device according to claim 1, comprising:
(a) a central region; and
(b) an enveloping material surrounding the central region, said enveloping
material being thermally insulating and permeable for the active component, wherein the central region is an inert support that is covered or coated with a first layer comprising a first sublayer, optionally a second sublayer and optionally a third sublayer, wherein said first layer being selected from the group consisting of:
(i) a first sublayer comprising a thermo-sensitive polymeric material, a pharmaceutically or biologically active component and an infrared absorbing compound and optionally an second sublayer, wherein said second sublayer is permeable for said pharmaceutically or biologically active component;
(ii) a first sublayer comprising a pharmaceutically or biologically active component and a second sublayer comprising a thermo-sensitive polymeric material and an infrared absorbing compound, wherein said second sublayer is optionally surrounded by a third sublayer, said third sublayer being permeable for said pharmaceutically or biologically active component;
(iii) a first sublayer comprising a pharmaceutically or biologically active component and a infrared absorbing compound and a second sublayer comprising the thermo-sensitive polymeric material, wherein said second sublayer is optionally surrounded by a third sublayer, said third sublayer being permeable for said pharmaceutically or biologically active component; and
(iv) a first sublayer comprising the infrared absorbing compound and an second sublayer comprising the thermo-sensitive polymeric material and the pharmaceutically or biologically active component, wherein said second sublayer is optionally surrounded by a third sublayer, said third layer being permeable for said pharmaceutically or biologically active component.

9. Drug delivery device according to claim 7, wherein the enveloping material is selected from the group consisting of hollow extended articles having a wall portion such as a catheter, said wall portion comprising the drug delivery device of claim 7.

10. Drug delivery device according to claim 8, wherein the enveloping material is selected from the group consisting of hollow extended articles having a wall portion such as a catheter, said wall portion comprising the drug delivery device of claim 8.

11. Drug delivery device according to claim 7, wherein the enveloping material is a surgical mesh or a part of a surgical mesh, said surgical mesh comprising the drug delivery device of claim 7.

12. Drug delivery device according to claim 8, wherein the enveloping material is a surgical mesh or a part of a surgical mesh, said surgical mesh comprising the drug delivery device of claim 8.

13. Drug delivery device according to claim 7, wherein the enveloping material is formed by the surface of the outer layer, said surface or a part thereof having indentations.

14. Drug delivery device according to claim 8, wherein the enveloping material is formed by the surface of the outer layer, said surface or a part thereof having indentations.

15. Drug delivery device according to claim 7, wherein the enveloping material comprises a porous weblike material.

16. Drug delivery device according to claim 8, wherein the enveloping material comprises a porous weblike material.

17. Drug delivery device according to claim 7, wherein the enveloping material is a hydrogel.

18. Drug delivery device according to claim 8, wherein the enveloping material is a hydrogel.

19. Drug delivery device according to claim 1, wherein the pharmaceutically or biologically active compound is selected from the group consisting of medicaments, diagnostic agents and contrast media for imaging.

20. Drug delivery device according to claim 17, wherein the medicament is selected from the group consisting of chemotherapeutic agents, analgesics, anaesthetics, hormonal substances, anti-microbial agents and antiarrhythmatic agents.

21. Drug delivery device according to claim 1, wherein the infrared absorbing compound is selected from the group consisting of phthalocyanines, naphthalocyanines, rylenes (perylenes, terrylenes, quaterrylenes), (organo)metal complexes, azo dyes, anthraquinones, squaric acid derivatives, immonium dyes, polymethines and derivatives thereof, polyanilines, carbon black, carbon nanotubes, carbon cages and carbon rods.

22. A method for releasing an active compound from a drug delivery device, the drug delivery device comprising a thermo-sensitive polymeric material, a pharmaceutically or biologically active component and an infrared absorbing compound, wherein the drug delivery device is exposed to infrared irradiation thereby crossing a phase transition and wherein said infrared absorbing compound comprises carbon.

23. The method according to claim 22, wherein the phase transition is a glass transition, a crossing of the LCST or a melt transition.

24. The method according to claim 22, wherein the infrared irradiation is supplied externally with respect to the drug delivery device.

25. The method according to claim 22, wherein the release characteristics of the thermo-sensitive polymeric material is modified remotely.

26. The method according to claim 22, wherein the drug delivery device is for implantation into a mammalian body.

27. The method according to claim 22, wherein the drug delivery device is pulse-wise or intermittently exposed to infrared irradiation.

28. The method according to claim 27, wherein the pharmaceutically or biologically active component comprises an analgesic or an anti-inflammatory drug.
